(19) United States
(12) Patent Application Publication
(10) Pub. No.: US 2010/0168449 A1
(43) Pub. Date: Jul. 1, 2010

(54) Spray dried zeolite catalyst

(76) Inventors: Roger A. Grey, West Chester, PA (US); Bernard Cooker, Malvern, PA (US); Edrick Morales, West Chester, PA (US)

Correspondence Address: LyondellBasell Industries, 3801 WEST CHESTER PIKE, NEWTOWN SQUARE, PA, 19073, US

(21) Appl. No.: 12/317,749

(22) Filed: Dec. 29, 2008

(51) Int. Cl. B01J 29/04; C07D 301/03

(52) U.S. Cl. 549/523; 502/64

(57) ABSTRACT

An attrition-resistant catalyst is prepared contacting a spray dried zeolite with a modifying agent. The modifying agent is (i) a halogen-free compound hydrolyzable to an oxide selected from the group consisting of silica, alumina, titania, zirconia, niobia, and mixtures thereof; or (ii) a sol selected from the group consisting of silica, alumina, titania, zirconia, niobia, and mixtures thereof.

### FIELD OF THE INVENTION

The invention relates to a method of preparing an attrition-resistant spray dried zeolite catalyst and its applications.

### BACKGROUND OF THE INVENTION

Zeolites may be used to catalyze many chemical transformations. See “Chapter 2. Catalyst Materials, Properties and Preparations” in Fundamentals of Industrial Catalytic Processes, C. H. Batholomew and R. J. Farrauto, Wiley Interscience (2006), pp. 60-117. The spray drying method has been used to form zeolites into microspheres. Spray dried zeolite catalysts are often used in fluidized bed or slurry processes. One of the problems with these processes is that the catalyst particles tend to attrit during use (U.S. Pat. Nos. 3,957,689, 4,276,196, 4,325,847, 4,569,833, 4,977,122, 5,221,648, and 6,710,003). Catalyst attrition can cause operational difficulties, e.g., in separation of catalyst from a liquid reaction mixture by filtration. It is desirable to produce attrition-resistant spray dried zeolite catalysts.

### SUMMARY OF THE INVENTION

The invention is a catalyst preparation method comprising contacting a spray dried zeolite with a modifying agent. The modifying agent is (i) a halogen-free compound hydrolyzable to an oxide selected from the group consisting of silica, alumina, titania, zirconia, niobia, and mixtures thereof; or (ii) a sol selected from the group consisting of silica, alumina, titania, zirconia, niobia, and mixtures thereof. The invention also includes: a catalyst prepared by the above method, an epoxidation process comprising reacting an olefin and hydrogen peroxide in the presence of a transition metal zeolite catalyst prepared by the method of the invention; and a direct epoxidation process comprising reacting an olefin, hydrogen, and oxygen in the presence of a noble metal and a transition metal zeolite catalyst prepared by the method of the invention.

### DETAILED DESCRIPTION OF THE INVENTION

In one aspect, the invention is a catalyst preparation method comprising contacting a spray dried zeolite with a modifying agent, wherein the modifying agent is (i) a halogen-free compound hydrolyzable to an oxide selected from the group consisting of silica, alumina, titania, zirconia, niobia, and mixtures thereof; or (ii) a sol selected from the group consisting of silica, alumina, titania, zirconia, niobia, and mixtures thereof.

In another aspect, the invention is a catalyst prepared by the above method.

The spray dried zeolite is prepared by spray drying a solution, a suspension, or a paste containing a zeolite. Zeolites are porous crystalline solids with well-defined structures. Generally they contain one or more of Si, Ge, Al, B, P, or the like, in addition to oxygen. Many zeolites occur naturally as minerals and are extensively mined in many parts of the world. Others are synthetic and are made commercially for specific uses. Zeolites can catalyze chemical reactions which take place mostly within the internal cavities of the zeolites. See “Chapter 2. Catalyst Materials, Properties and Preparations” in Fundamentals of Industrial Catalytic Processes, C. H. Batholomew and R. J. Farrauto, Wiley Interscience (2006), pp. 60-117.

Transition metal zeolites may be used. Transition metal zeolites are zeolites comprising transition metals in the framework. A transition metal is a Group 3-12 element. The first row of transition metals are from Sc to Zn. Preferred transition metals are Ti, V, Mn, Fe, Co, Cr, Zr, Nb, Mo, and W. More preferred are Ti, V, Mo, and W. Titanium zeolites are particularly preferred.

Preferred titanium zeolites are titanium silicates (titanosilicates). Preferably, they contain no element other than titanium, silicon, and oxygen in the lattice framework (see R. Szostak, “Non-aluminosilicate Molecular Sieves,” in Molecular Sieves: Principles of Synthesis and Identification (1989), Van Nostrand Reinhold, pp. 205-82). Small amounts of impurities, e.g., boron, iron, aluminium, phosphorous, copper, and the like, and mixtures thereof, may be present in the lattice. The amount of impurities is preferably less than 0.5 weight percent (wt %), more preferably less than 0.1 wt %. Preferred titanium silicates will generally have a composition corresponding to the following empirical formula: xTiO₂.(1−x)SiO₂, where x is between 0.0001 and 0.5000. More preferably, the value of x is from 0.01 to 0.125. The molar ratio of Si to Ti in the lattice framework of the zeolite is advantageously from 9.5:1 to 99:1, most preferably from 9.5:1 to 60:1. Particularly preferred titanium silicates are titanium silicalites (see Catal. Rev.-Sci. Eng., 39(3) (1997) 209). Examples of titanium silicalites include TS-1 (titanium silicalite-1, a titanium silicalite having an MFI topology analogous to that of the ZSM-5 aluminosilicate), TS-2 (having an MEL topology analogous to that of the ZSM-11 aluminosilicate), and TS-3 (as described in Belgian Pat. No. 1,001,038). Titanium silicates having framework structures isomorphous to zeolite beta, mordenite, ZSM-12, MCM-22, MCM-41, and MCM-48 are also suitable for use. Examples of MCM-22, MCM-41, and MCM-48 zeolites are described in U.S. Pat. Nos. 4,954,325, 6,077,498, and 6,114,551; Maschmeyer, T., et al., Nature 378(9) (1995) 159; Tanev, P. T., et al., Nature 368 (1994) 321; Corma, A., J. Chem. Soc., Chem. Commun. (1998) 579; Wei, D., et al., Catal. Today 51 (1999) 501); Wu, P., et al., Chem. Lett. (2000) 774; and J. Phys. Chem. 105 (2001) 2897. TS-1 and Ti-MCM-22 are particularly preferred.

A zeolite is generally prepared in the presence of an organic templating agent (see, e.g., U.S. Pat. No. 6,849,570). Suitable templating agents include alkyl amines, quaternary ammonium compounds, etc. When a zeolite is crystallized, it usually contains organic templating agent within its pores. Zeolites containing templating agents may be spray dried to produce the catalyst of the invention without being calcined first. Alternatively, a zeolite is calcined in an oxygen-containing atmosphere to remove the templating agent before it is spray dried.

Generally, the spray dried zeolite comprises a binder. A binder helps to improve the mechanical strength and/or the physical properties of the spray dried zeolite (e.g., crushing strength, surface area, pore size, pore volume). Sometimes they modify the chemical properties (e.g., acidity, basicity) of the zeolite and its catalytic activity. Generally the binder constitutes from 1 to 90 wt %, preferably 2 to 60 wt %, more preferably from 5 to 50 wt % of the catalyst. The concentration of the binder is defined as the weight percent of the non-zeolitic component of the spray dried zeolite after the particles are calcined in an oxygen-containing atmosphere to remove the organic components.

Suitable binders include silica, titania, alumina, zirconia, magnesia, silica-alumina, montmorillonite, kaolin, bentonite, halloysite, dickites, nacrite, and anauxite, and the like, and mixtures thereof. Examples of clays can be found in “Chapter 2. Clay as Potential Catalyst Material,” Zeolite, Clay, and Heteropoly Acid in Organic Reactions (1992) Kodansha Ltd., Tokyo. Preferred binders include silica, titania, alumina, and mixtures thereof. Silica is particularly preferred.

One preferred method for preparing a suspension suitable for the spray drying operation is to mix the zeolite, a sol, and optionally additional solvent. A sol is a colloidal suspension of solid particles in a liquid. In a sol, the thermal energy keeps the colloidal particles under constant and random agitation known as Brownian motion. This thermal driving force must be of a magnitude larger than the action of gravity, which means that each particle must have a very small mass. Colloidal particles are usually spherical or nearly spherical. Their sizes depend on the nature of the material, typically are <0.2 μm with metal or non-metal oxides. See Pierre, A. C., “Sol-Gel Technology,” Kirk-Othmer Encyclopedia of Chemical Technology, on-line edition (2008). A sol comprises a collection of small particles of the binder in hydrated form.

A sol may be prepared by mixing the binder or a binder precursor with a solvent. A binder precursor is a compound that can be converted to the binder during spray drying and/or calcination. Examples of suitable silica precursors include silicon halide (e.g., tetrachlorosilicate), tetraalkoxysilicate (tetramethoxysilicate, tetraethoxysilicate, tetraisopropoxysilicate, and the like). Examples of suitable titania precursors include titanium chloride, titanium sulfate, titanyl sulfate, titanyl oxosulfate, titanium tetramethoxide, titanium tetraethoxide, titanium tetraisopropoxide, titanium tetraisobutoxide, titanium tetratertbutoxide, titanium tetraphenoxide, titanium phenoxytrichloride, titanium triphenoxychloride, titanium acetylacetonate, titanium ethoxytrifluoride, titanium ethoxytrichloride, titanium ethoxytribromide, titanium diethoxydifluoride, titanium diethoxydichloride, titanium diethoxydibromide, titanium triethoxyfluoride, titanium triethoxychloride, titanium isobutoxytrichloride, and titanium diisobutoxydichloride. Examples of suitable alumina precursors include aluminium chloride, aluminium sulfate, aluminium acetate, aluminium trimethoxide, aluminium triethoxide, aluminium triisopropoxide, and aluminium triisobutoxide. Suitable solvents for making a sol include water, alcohols, amides, nitriles, and the like, and mixtures thereof. Preferred solvents are water, alcohols, and their mixtures.

If a binder precursor is used, a hydrolyzing agent, e.g., water, an acid, or base is used to hydrolyze the binder precursor to prepare the sol. Suitable acids include hydrochloric acid, hydrobromic acid, sulfuric acid, nitric acid, phosphoric acids, and carboxylic acids (e.g., formic acid, acetic acid, benzoic acid). Suitable bases useful as hydrolyzing agents include, e.g., sodium hydroxide, ammonium hydroxide, alkylammonium hydroxides, ammonia, alkylamines, sodium carbonate, and sodium bicarbonate. Organic acids and bases are preferred hydrolyzing agents because they do not introduce hard-to-remove metal cations or inorganic anions. Commercially available silica sols such as Ludox® AS40 or Ludox® HS40 from Grace Davison, and Nalco® 2350, Nalco® 2360, Nalco® 2326, Nalco® 2398 from Nalco Company may be used.

The suspension suitable for spray drying typically contains from 50 to 90 wt % solvent, from 1 to 40 wt % zeolite, and from 1 to 40 wt % binder. The amount of zeolite to the binder is typically in the range of 95:5 to 5:95 in weight, preferably from 9:1 to 1:1.

Spray drying method is known in forming zeolites. See U.S. Pat. Nos. 4,954,653, 4,701,428, 5,500,199, 6,524,984, and 6,106,803. Generally a spray dryer is used. A spray dryer is usually a large vertical chamber through which hot gas is blown and into which a solution, a suspension, or a pumpable paste is sprayed by a suitable atomizer. Particles produced by spray drying are generally from 5 μm to 1 mm in diameter. During spray-drying, the suspension is first broken down into fine droplets by an atomizing device, which are then fluidized and dried in a process gas (also called drying gas). See Maters, K, Spray Drying In Practice, SprayDryConsultant International ApS (2002) pp. 1-15. Suitable atomizing devices are, for example, single-fluid pressure nozzles, two-fluid atomization nozzles, or rotary atomizers. The inlet temperature of the process gas may be between 100 and 700° C., preferably between 150 and 500° C.; the exit temperature of the process gas may be between 50 and 200° C., preferably between 80 and 160° C. The sprayed droplets are dried by the process gas to produce spray dried zeolite. The process gas and the droplets being spray dried may be passed in the same or opposite directions.

The spray dried zeolite may be calcined. Generally, the calcination of spray dried zeolite can be carried out at a temperature of 200 to 1000° C., preferably of 400 to 700° C. Calcination may be performed in an inert gas. Nitrogen is one preferred inert gas. In one preferred method, the spray dried zeolite are calcined in a nitrogen atmosphere first, then in an oxygen-containing atmosphere to burn off any organic residue.

The spray dried zeolite is contacted with a modifying agent. The modifying agent for the present invention may be a halogen-free compound hydrolyzable to an oxide selected from the group consisting of silica, alumina, titania, zirconia, niobia, and mixtures thereof. Any chemical compound that can react with water at a temperature of 20 to 200° C. in the presence of an acid or base to form an oxide selected from the group consisting of silica, alumina, titania, zirconia, niobia, and mixtures thereof may be used. Suitable modifying agents include tetraalkoxysilicates, titanium(IV) alkoxides, titanium carboxylates, aluminium alkoxides, zirconium alkoxides, niobium alkoxides, and the like. Example of suitable modifying agents include tetramethoxysilicate, tetraethoxysilicate, tetraisopropoxysilicate, titanium tetramethoxide, titanium tetraethoxide, titanium tetraisopropoxide, titanium tetraisobutoxide, titanium tetra-tert-butoxide, titanium tetraphenoxide, aluminium acetate, aluminium trimethoxide, aluminium triethoxide, aluminium triisopropoxide, aluminium triisobutoxide, zirconium tetramethoxide, zirconium tetraethoxide, zirconium tetraisopropoxide, zirconium tetraisobutoxide, zirconium tetra-tert-butoxide, zirconium tetraphenoxide, niobium acetate, niobium pentamethoxide, niobium pentaethoxide, niobium pentaisopropoxide, and niobium pentaisobutoxide. Preferred modifying agents include tetraalkoxysilicates, aluminum alkoxides, titanium alkoxides, and mixtures thereof.

The modifying agent for the present invention may also be a sol selected from the group consisting of silica, alumina, titania, zirconia, niobia, and mixtures thereof. Silica, alumina, titania, zirconia, and niobia sols described in the previous sections may be used as modifying agents. Silica, alumina, and titania sols are preferred.

Many suitable methods may be used to contact the spray dried zeolite with the modifying agent. Incipient wetness is one preferred method. For example, tetraethoxysilicate may be added directly to the spray-died particles.

Alternatively, a mixture of a modifying agent and a solvent may be used. Any solvent that can mix with the modifying agent may be used, e.g., alkanes, aromatic solvents, alcohols, ethers, ester, water, and mixtures thereof.

The temperature at which the spray dried zeolite is contacted with the modifying agent is not critical. Conveniently, it is performed at 10 to 100° C.

The catalyst prepared in accordance with the present invention has improved attrition resistance as compared with the spray dried zeolite. Although not bound by any theory, this may be due to that the modifying agent fills the cracks, crevices, gaps, or voids, or coats the outside surfaces of the spray dried zeolite.

The catalyst is preferably further calcined. Generally, the calcination of the catalyst is carried out at a temperature of 200 to 1000° C., preferably of 400 to 700° C. Calcination may be performed in an inert gas. Nitrogen is one preferred inert gas. In one preferred method, the spray dried zeolite are calcined in a nitrogen atmosphere first, then in an oxygen-containing atmosphere to burn off any organic residue.

The catalysts prepared in accordance with the present invention may be used in many chemical reactions, including, cracking, alkylation, isomerization, oxidation, and the like. See “Chapter 2. Catalyst Materials, Properties and Preparations” in Fundamentals of Industrial Catalytic Processes, C. H. Batholomew and R. J. Farrauto, Wiley Interscience (2006), pp 60-117; New Developments in Selective Oxidation, G. Centi and F. Trifiro, Ed., pp. 33-38

In yet another aspect, the invention is an epoxidation process comprising reacting an olefin and hydrogen peroxide in the presence of a catalyst comprising a transition metal zeolite prepared by the method of the invention.

Preferably the catalyst comprising the transition metal zeolite is calcined before it is used in the epoxidation. The calcination may be performed in an inert gas or an oxygen-containing atmosphere. Nitrogen is one preferred inert gas. In one preferred method, the catalyst is calcined first in a nitrogen atmosphere, then in an oxygen-containing atmosphere to burn off any organic residue. The calcination may be performed at a temperature of 200 to 1000° C., preferably at 300 to 700° C.

The epoxidation process uses an olefin. Suitable olefins include any olefin having at least one carbon-carbon double bond, and generally from 2 to 60 carbon atoms. Preferably the olefin is an acyclic alkene of from 2 to 30 carbon atoms; the process is particularly suitable for epoxidizing C₂-C₆ olefins. More than one double bond may be present in the olefin molecule, as in a diene or triene. The olefin may be a hydrocarbon or may contain functional groups such as halogen, carboxyl, hydroxyl, ether, carbonyl, cyano, or nitro groups, or the like. In a particularly preferred process, the olefin is propylene and the epoxide is propylene oxide

The epoxidation process uses hydrogen peroxide. Preferably a solution of hydrogen peroxide in a solvent is used. Suitable solvents are liquid under the reaction conditions. They include, for example, oxygen-containing hydrocarbons such as alcohols, nitriles such as acetonitrile, carbon dioxide, and water. Suitable oxygenated solvents include alcohols, ethers, esters, ketones, carbon dioxide, water, and the like, and mixtures thereof. Preferred oxygenated solvents include aliphatic C₁-C₄ alcohols such as methanol, ethanol, isopropanol, and tert-butanol, their mixtures, and mixtures of these alcohols with water.

The amount of olefin used relative hydrogen peroxide is not very critical. Generally an olefin to hydrogen peroxide molar ratio of 1:10 to 10:1 is used.

It is advantageous to work at a pressure of from 15 to 3,000 psig. The process is carried out at a temperature effective to achieve the desired olefin epoxidation, preferably at temperatures in the range of 0 to 200° C., more preferably, 20 to 150° C.

In yet another aspect, the invention is a direct epoxidation process comprising reacting an olefin, hydrogen, and oxygen in the presence of a noble metal and a catalyst comprising a transition metal zeolite prepared by the method of the invention.

The direct epoxidation process is performed in the presence of a noble metal. Suitable noble metals include gold, silver, platinum, palladium, iridium, ruthenium, osmium, rhenium, rhodium, and mixtures thereof. Preferred noble metals are Pd, Pt, Au, Re, Ag, and mixtures thereof. Palladium, gold, and their mixtures are particularly desirable.

There are no particular restrictions regarding the choice of the noble metal compound or complex used as the source of the noble metal. Suitable compounds include nitrates, sulfates, halides (e.g., chlorides, bromides), carboxylates (e.g., acetate), and amine or phosphine complexes of noble metals (e.g., palladium(II) tetraammine bromide, tetrakis(triphenylphosphine) palladium(0)).

The weight ratio of the transition metal zeolite to noble metal is not particularly critical. However, a transition metal zeolite to noble metal weight ratio of from 10:1 to 5,000:1 (grams of transition metal zeolite per gram of noble metal) is preferred.

The method in which the noble metal is incorporated in the direct epoxidation process is not critical. The noble metal may be added to the zeolite or a carrier. Suitable carriers for the supported noble metal include carbon, titania, zirconia, niobia, silica, alumina, silica-alumina, titania-silica, zirconia-silica, niobia-silica, ion-exchange resin, and the like, and mixtures thereof.

The direct epoxidation process uses an olefin. Suitable olefins for the epoxidation process described in the previous section are applicable to the present direct epoxidation process.

The direct epoxidation process uses oxygen and hydrogen. The molar ratio of hydrogen to oxygen can usually be varied in the range of H₂:O₂=1:100 to 5:1 and is especially favorable at 1:5 to 2:1. The molar ratio of oxygen to olefin is usually 1:1 to 1:20, and preferably 1:1.5 to 1:10. Air may be used as a source of oxygen.

The direct epoxidation process preferably uses an inert gas, in addition to the olefin, oxygen, and hydrogen. Any desired inert gas can be used. Suitable inert gases include nitrogen, helium, argon, and carbon dioxide. Saturated hydrocarbons with 1-8, especially 1-6, and preferably 1-4 carbon atoms, e.g., methane, ethane, propane, and n-butane, are also suitable. Nitrogen and saturated C₁-C₄ hydrocarbons are preferred inert gases. Mixtures of inert gases can also be used. The molar ratio of olefin to gas is usually in the range of 100:1 to 1:10 and especially 20:1 to 1:10.

The direct epoxidation process may be performed in a continuous flow, semi-batch, or batch mode. A continuous flow process is preferred.

The direct epoxidation process is generally carried out at a pressure of from 15 to 3,000 psig. The process is carried out at a temperature effective to achieve the desired olefin epoxidation, preferably at temperatures in the range of 0-200° C., more preferably, 20-150° C. Preferably, at least a portion of the reaction mixture is a liquid under the reaction conditions.

The direct epoxidation process preferably uses a reaction solvent. Suitable reaction solvents are liquid under the reaction conditions. They include, for example, oxygen-containing hydrocarbons such as alcohols, nitrites such as acetonitrile, carbon dioxide, and water. Suitable oxygenated solvents include alcohols, ethers, esters, ketones, carbon dioxide, water, and the like, and mixtures thereof. Preferred oxygenated solvents include aliphatic C₁-C₄ alcohols such as methanol, ethanol, isopropanol, tert-butanol, their mixtures, and mixtures of these alcohols and water.

### EXAMPLE 1

### Spray Dried Silica-Bound TS-1 Modified With Tetraethoxysilicate (Catalyst A)

Preparation

A TS-1 (2 wt % Ti) is prepared by following procedures disclosed in U.S. Pat. Nos. 4,410,501 and 4,833,260.

A spray dried silica-bound TS-1 (containing 20 wt % binder) is prepared from TS-1 by following procedures disclosed in U.S. Pat. Appl. Pub. No. 20070027347 with the exception that zinc oxide is not used.

Into a 100-mL beaker containing 21.6 g silica-bound spray dried TS-1 (not calcined; containing 7.3 wt % Ti, 33 wt % Si, and 11 wt % C; mean particle diameter, 30 micron), a sample of tetraethoxysilicate (16.3 g) is added at about 20° C. in 0.5-g doses with mixing over a 40-min period until the solids achieve incipient wetness. The solids are heated at 120° C. in an oven for 24 h with a 5 mol % oxygen-in-nitrogen purge. The solids are then calcined in air in a static furnace. The temperature is raised from 23 to 110° C. at a rate of 10° C./min, held for 4 h, then raised from 110° C. to 550° C. at a rate of 2° C./min, and finally held for 4 h at 550° C. The final product (Catalyst A) contains 1.7 wt % Ti, 45 wt % Si, and <0.1 wt % C.

Propylene Epoxidation

A stock solution of 5 wt % hydrogen peroxide in methanol is prepared by slowly adding 150 g of 30 wt % aqueous hydrogen peroxide to 761 g of reagent grade methanol with mixing.

The epoxidation is conducted by charging a 100-mL stainless steel pressure reactor with 40 g of the above hydrogen peroxide stock solution and 0.15 g Catalyst A, and 20 g propylene. The reactor is immersed in a preheated bath to bring the reactor to 50° C. and the reaction is stirred at 50° C. for 30 min. The reactor is cooled to 23° C. in an ice bath and the gases vented into a gas bag for gas chromatography (GC) analyses. The liquid is recovered and analyzed by GC for the oxygenated products derived from propylene including, propylene oxide (PO), propylene glycol, and propylene glycol methyl ethers. The hydrogen peroxide remaining in solution is determined by titration and liquid chromatography (LC) analyses. PO selectivity is the moles of PO formed in the reaction divided by the moles of hydrogen peroxide consumed. The epoxidation results are shown in Table 1.

Attrition Resistance Test

A slurry containing Catalyst A (10 g) and 190 g of de-ionized water is placed in a Waring blender (Model 700g available from Fisher, Fisher Catalog #14-509-10) and blended for 30 min at a speed of 22,000 rpm with a 1-L heat-resistance borosilicate container. The temperature of the slurry is 20° C. at the start of the test and rises to 55° C. after 15 min. The slurry is collected with a pipette and transferred to a Millipore 340-mL pressure filter holder (Model XX40 047 00) equipped with a Millipore 0.45-μm filter paper. The filtration is performed under a 5 psig differential pressure. The amount of filtrate collected after 15 min is 21.2 mL.

The amount of filtrate collected for a catalyst at a given period of time is a measure of the attrition resistance of the catalyst. A stronger catalyst is less likely to attrit to form smaller particles during the blending. As a result the catalyst filters faster due to fewer blockages of filter paper pores.

### EXAMPLE 2

### Spray Dried Silica-Bound TS-1 Modified With Tetrabutoxytitanate (Catalyst B)

Preparation

Into a 100-mL beaker containing 20 g spray dried silica-bound TS-1 (mean particle diameter, 35 μm) prepared from a non-calcined TS-1, a sample of tetrabutoxytitanate (16.3 g) is added at about 20° C. in 0.5-g doses with mixing over a 40-min period until the solids achieve incipient wetness. The solids are heated and calcined by following the procedure of Example 1. The final product (Catalyst B) contains 12 wt % Ti, 35 wt % Si, and <0.1 wt % C.

The propylene epoxidation and attrition resistance test procedures of Example 1 are repeated, except that Catalysts B is used. The results are shown in Table 1.

### EXAMPLE 3

### Spray Dried Silica-Bound TS-1 Modified With Tetrabutoxytitanate (Catalyst C)

Preparation

Into a 100-mL beaker containing 25 g spray dried silica-bound TS-1 (20 wt % binder; calcined in air at 600° C.; mean particle diameter, 35 μm), a sample of tetrabutoxytitanate (19.8 g) is added at about 20° C. in 0.5-g doses with mixing over a 40-min period until the solids achieve incipient wetness. The solids are heated and calcined by following the procedure of Example 1. The final product (Catalyst C) contains 10 wt % Ti, 35 wt % Si, and <0.1 wt % C.

The propylene epoxidation and attrition resistance test procedures of Example 1 are repeated, except that Catalysts C is used. The results are shown in Table 1.

### EXAMPLE 4

### Spray Dried Titania-Bound TS-1 Modified With Tetrabutoxytitanate (Catalyst D)

Preparation

A spray dried silica-bound TS-1 (containing about 20 wt % binder) is prepared from a TS-1 (2 wt % Ti) by following the procedure of Example 1 of co-pending application Ser. No. 12/011,659 filed Jan. 29, 2008.

Into a 100-mL beaker containing 21.4 g spray dried titania-bound TS-1 (calcined in air at 600° C.; mean particle diameter, 35 μm), a sample of tetrabutoxytitanate (45.7 g) is added at about 20° C. in 0.5-g doses with mixing over a 40-min period until the solids achieve incipient wetness. The solids are heated and calcined by the following the procedure of Example 1. The final product (Catalyst D) contains 30 wt % Ti, 23 wt % Si, and <0.1 wt % C.

The propylene epoxidation and attrition resistance test procedures of Example 1 are repeated, except that Catalysts D is used. The results are shown in Table 1.

### EXAMPLE5

### Spray Dried Titania-Bound TS-1 Modified With Tetraethoxysilicate (Catalyst E)

Into a 100-mL beaker containing 25 g spray dried titania-bound TS-1 (20 wt % binder; calcined in air at 600° C.; mean particle diameter, 30 μm), a sample of tetraethoxysilicate (43.8 g) is added at about 20° C. in 0.5-g doses with mixing over a 40-min period until the solids achieve incipient wetness. The solids are heated and calcined by the following the procedure of Example 1. The final product (Catalyst E) contains 6.5 wt % Ti, 45 wt % Si, and <0.1 wt % C.

The propylene epoxidation and attrition resistance test procedures of Example 1 are repeated, except that Catalysts E is used. The results are shown in Table 1.

### COMPARATIVE EXAMPLE 6

### Spray Dried Silica-Bound TS-1 Without Modification (Catalyst F)

Preparation

A spray dried silica-bound TS-1 is prepared by following the procedure of Example 1 of co-pending application Ser. No. 12/011,659 filed Jan. 29, 2008. The product (Catalyst F) contains about 20 wt % silica binder and 80 wt % TS-1 (2 wt % Ti). Catalyst F is calcined in air at 600° C.

The propylene epoxidation and attrition resistance test procedures of Example 1 are repeated, except that Catalysts F is used. The results are shown in Table 1.

TABLE 1

| | Example | | | | | |
|---|---|---|---|---|---|---|
| | 1 | 2 | 3 | 4 | 5 | C. 6 |
| Catalyst | A | B | C | D | E | F |
| Epoxidation Results | | | | | | |
| H2O2 conversion (%) | 75 | 69 | 65 | 77 | 72 | 89 |
| PO Selectivity (%) | 97 | 97 | 97 | 96 | 96 | 96 |
| Attrition and Filtration Test | | | | | | |
| Filtration (mL/15 min) | 21.2 | 34.5 | 35.1 | 32.5 | 99.8 | 14.5 |

### EXAMPLE 7

### Preparation of Catalyst G

A sample of Catalyst E in Example 5 (16 g) is impregnated with an aqueous palladium tetraammine dinitrate solution (5.37 wt % Pd) at 30° C. The slurry pH is adjusted to 7.6. The solids are filtered, dried, then calcined at 300° C. in air for 3 h. The calcined solids are transferred to a quartz tube and treated with a 4 volume percent (vol %) hydrogen-in-nitrogen stream (100 mL/h) at 100° C. for 3 h. The material obtained (Catalyst G) is expected to contain about 0.1 wt % Pd.

### COMPARATIVE EXAMPLE 8

### Preparation of Catalyst H

A sample of Catalyst F in Example 6 (16 g) is impregnated with an aqueous palladium tetraammine dinitrate solution (5.37 wt % Pd) at 30° C. The slurry pH is adjusted to 7.6. The solids are filtered, dried, then calcined at 300° C. in air for 3 h. The calcined solids are transferred to a quartz tube and treated with a 4 vol % hydrogen-in-nitrogen stream (100 mL/h) at 100° C. for 3 h. The material obtained (Catalyst H) is expected to contain about 0.1 wt % Pd.

### EXAMPLE 9

### Direct Propylene Epoxidation With Catalyst G

An ammonium dihydrogen phosphate solution is prepared by dissolving ammonium dihydrogen phosphate (5.75 g) in de-ionized water (250 g) and methanol (750 g).

A 300-mL stainless steel reactor is charged with Catalyst G (3.0 g) and ammonium dihydrogen phosphate solution prepared above (100 mL). The slurry in the reactor is heated to 50° C. under about 300 psig, and is stirred at 800 rpm. Additional ammonium dihydrogen phosphate solution is pumped to the reactor at a rate of about 50 g/h. The feed gas flow rates are about 4500 sccm (standard cubic centimeters per minute) for 5 vol. % oxygen in nitrogen, 280 sccm for propylene, and 110 sccm for hydrogen. The pressure in the reactor is maintained at 300 psig via a back pressure regulator with the feed gases pass continuously through the reactor. The gaseous effluent is analyzed by an on-line GC. The liquid is analyzed by an off-line GC periodically. The products are expected to be propylene oxide, propane, and derivatives of propylene oxide such as propylene glycol, propylene glycol monomethyl ethers, dipropylene glycol, and dipropylene glycol methyl ethers.

### COMPARATIVE EXAMPLE 10

### Direct Propylene Epoxidation With Catalyst H

The procedure of Example 9 is repeated, except that Catalysts H is used.

It is expected that the attrition resistance of Catalyst G is better than that of Catalyst H. The improvement may be shown by a filtration test (as described in Example 1) of the reaction mixture at the end of the direct epoxidation.

We claim:

1. A catalyst preparation method comprising contacting a spray dried zeolite with a modifying agent to form the catalyst, wherein the modifying agent is (i) a halogen-free compound hydrolyzable to an oxide selected from the group consisting of silica, alumina, titania, zirconia, niobia, and mixtures thereof; or (ii) a sol selected from the group consisting of silica, alumina, titania, zirconia, niobia, and mixtures thereof.

2. The method of claim 1 wherein the catalyst is calcined at a temperature of 200 to 1000° C.

3. The method of claim 1 wherein the spray dried zeolite comprises a binder.

4. The method of claim 1 wherein the modifying agent is a halogen-free compound hydrolyzable to an oxide selected from the group consisting of silica, alumina, titania, zirconia, niobia, and mixtures thereof.

5. The method of claim 1 wherein the zeolite is a transition metal zeolite.

6. The method of claim 1 wherein the zeolite is a titanium zeolite.

7. A catalyst prepared by contacting a spray dried zeolite with a modifying agent, wherein the modifying agent is (i) a halogen-free compound that may hydrolyzable to an oxide selected from the group consisting of silica, alumina, titania, zirconia, niobia, and mixtures thereof; or (ii) a sol selected from the group consisting of silica, alumina, titania, zirconia, niobia, and mixtures thereof.

8. The catalyst of claim 7 calcined at a temperature from 200 to 1000° C.

9. The catalyst of claim 7 wherein the spray dried zeolite comprises a binder.

10. The catalyst of claim 7 wherein the modifying agent is a halogen-free compound hydrolyzable to an oxide selected from the group consisting of silica, alumina, titania, zirconia, niobia, and mixtures thereof.

11. The catalyst of claim 7 wherein the zeolite is a transition metal zeolite.

12. An epoxidation process comprising reacting with an olefin and hydrogen peroxide in the presence of the catalyst of claim 11 to produce an epoxide.

13. The process of claim 12 wherein the catalyst is calcined at a temperature from 200 to 1000° C.

14. A direct epoxidation process comprising reacting an olefin, hydrogen, and oxygen in the presence of the catalyst of claim 11 and a noble metal.

15. The process of claim 14 wherein the transition metal zeolite is a titanium zeolite.
